(19) United States
(12) Patent Application Publication
(10) Pub. No.: US 2005/0080322 A1
(43) Pub. Date: Apr. 14, 2005

(54) Monitoring method and monitoring system for assessing physiological parameters of a subject

(76) Inventor: Ronen Korman, Petach Tikva (IL)

Correspondence Address: Martin Moynihan;Anthony Castorina, Suite 207, 2001 Jefferson Davis Highway, Arlington, VA, 22202, US

(21) Appl. No.: 10/503,934

(22) PCT Filed: Mar. 18, 2002
(86) PCT No.: PCT/IL02/00214

(51) Int. Cl. A61B 5/00

(52) U.S. Cl. 600/300; 128/904

(57) ABSTRACT

A system for measuring at least one physiological parameter of the user, analyzing the parameter measured and providing feedback to the user. Analysis of at least one physiological parameter of the user may optionally and preferably be performed by a remotely located data processing server. The present invention enables such a measurement to preferably be used to directly generate medical information about the user, optionally performed by the sensing device or alternately by the remote server or alternately by the gateway. Such information may then optionally be sent to medical personnel, for example at a contact monitoring center and/or to any other location, through a gateway device. The gateway device is preferably a mobile gateway device and communicates with the preferred but exemplary wrist-mounted device of the present invention through a wireless communication channel.

### FIELD OF THE INVENTION

The present invention is of a method and a system for measuring at least one physiological parameter of a subject, and for relaying such information through a mobile gateway to a remote data processing server for analysis.

### BACKGROUND OF THE INVENTION

For medical reasons, human subjects may be monitored in a variety of ways. A number of these monitoring methods are non-invasive. For example, heart function can be monitored in a non-invasive manner in a human subject by using electrodes, which must be attached to the skin of the human subject. Heart rate and blood pressure can be monitored in a non-invasive manner by an inflating cuff. Temperature and respiration rate may also optionally be monitored non-invasively, also optionally together with some other physiological parameters.

Physiological conditions and physiological parameters characterizing these conditions are important factors in determining the state of health and the physical condition of an individual, both generally and in response to physical or emotional stress. Periodic monitoring of these physical parameters is particularly important for individuals having cardiac disease and/or reduced cardiac functioning, or high blood pressure. However, physically healthy individuals may also wish to monitor their heart rate and blood pressure periodically, particularly in stressful situations, for example when engaging in strenuous exercise.

Monitoring of the condition of a human subject could be very useful as part of the overall health maintenance of the human subject, and could be used in order to detect deterioration in the physiological condition of the subject before a concomitant deterioration in the health of the subject becomes noticeable. Examples of adverse physiological conditions which could be detected with regular non-invasive monitoring include but are not limited to excessive weight gain or loss; arrhythmia and other heart conditions; incipient diabetes in the form of improper glucose metabolism; and loss of lung capacity or other problems with respiration.

In order to support regular monitoring of the physiological condition of human subjects in their normal environment, such as in the home and at the office for example, the equipment must be non-invasive and easy to use. The equipment would then be able to monitor at least one physiological parameter of the user, without requiring the user to perform any complicated actions and/or to operate complex devices. (As used herein, the term “physiological data” means the actual “physiological signal” as measured by a sensor; “physiological parameter” refers to information which may be derived from the physiological data; and the term “physiological condition” means the overall condition of the subject wellness). Indeed, it would be highly preferred for the equipment to be incorporated as part of the regular daily living routine of the subject, since the requirement for any additional or special actions on the part of human subject is likely to result in decreased compliance. In addition, the equipment should be robust yet inexpensive. It would be further preferred that the measurement of physiological data, and the analysis thereof, would be conducted in a transparent manner to the user without requiring active user intervention or being inconvenient for the user.

There are a number of devices for measuring physiological data. Some of them are disclosed in U.S. Pat. No. 5,544,661 to Davis et al, U.S. Pat. No. 6,290,646 to Cosentino et al, and U.S. Pat. No. 3,926,179 to Petzke. Although non-invasive, such equipment is nevertheless bulky and uncomfortable for the human subject, who is attached to a network of cables and wired sensors. The use of these sensing devices is also not simple. They do not meet the requirement of being incorporated as part of the regular daily living routine of the subject.

Individuals who wish to monitor physiological conditions of their body, however, may not have the knowledge required for judging and interpreting physiological parameters and medical information indicating the current physical status of their health. As used herein, the term “medical information” refers to the information, which may be extracted or otherwise obtained by analyzing physiological signal or parameter and/or a combination of them. Frequent examinations by skilled medical personnel consume time and money, such that the human subjects may not have such examinations performed at a desirable frequency.

In order to overcome these hurdles methods of communicating physiological parameters to remotely located medical personnel have been developed. U.S. Pat. No. 5,772,586 to Heinonen et al, U.S. Pat. No. 5,840,020 to Heinonen et al and published PCT Application No. WO 90/08361 to Begun et al, teach methods of communicating physiological parameters, and in particular glucose level in the blood of a subject and ECG data to remotely located medical personnel. The methods disclosed require the permanent presence of qualified medical personnel at the remote location for manually analyzing the data received and providing feedback to a subject that transmitted the data. None of these references teach or suggest the automatic analysis of physiological parameters of the subject.

Heinonen discloses the use of a cellular telephone, but the teachings are quite restrictive, as they are only concerned with the transmission of the data. Another disadvantage of the methods disclosed by Heinonen is their reliance and dependence on a particular type of cellular telephone. It is an additional disadvantage of the method suggested by Heinonen is the fact that in order to transmit the data, the subject has to hold the phone and feed it with appropriate data. Furthermore, Heinonen has another major disadvantage in that the teachings are generally considered with transmission of the data through a telephone, but do not teach or suggest mobile monitoring of the subject.

U.S. Pat. No. 5,544,661 to Davis et al discloses a wireless data transmission system, in which the cellular telephone is part of the sensor arrangement itself. This significantly increases the costs of such an arrangement and makes its use limited. Furthermore, the sensor arrangement can only be described as “portable” in the sense that the electrodes and devices may be worn by the user. However, the use of such sensors cannot be automatic, as the user must attach electrodes to different points of the body before use. Therefore, the sensors are awkward and inconvenient, and cannot in any case provide continuous or near-continuous monitoring of one or more physiological functions of the user.

### SUMMARY OF THE INVENTION

The background art does not teach or suggest a system which can conveniently, non-intrusively, automatically and autonomously measure one, or more physiological parameters, in order to extract medical information such as heart rate, breathing rate, body temperature and blood pressure. The background art also does not teach or suggest such a system or method which can transmit the collected data to a remote data processing location, while maintaining data integrity. The background art also does not teach or suggest such a system which may invoke an alarm, if necessary, after receiving and analyzing the data.

The background art also does not teach or suggest such a system that provides true mobility and freedom for the subject wearing the sensing device. The background art also does not teach or suggest such a system that includes a mobile gateway communicating on one hand with a sensing device and on the other hand with a public terrestrial or wireless communication network.

The present invention overcomes these deficiencies of the background art by providing a system for monitoring at least one physiological parameter of the user. The present invention enables such a physiological parameter to preferably be transformed into medical information about the user, by analysis of physiological data. Such information may then optionally and preferably be transmitted through a gateway device to a remote data processing server, which more preferably serves qualified medical personnel at a contact center and/or at any remote location. Alternatively, raw physiological data may optionally be transmitted to the contact center for analyzing the raw data by medical personnel. More preferably, the raw data is processed by a data processing server, which then most preferably stores the results in a database. The server may optionally perform automatic diagnosis of the condition of the subject, and/or may optionally alert the subject or qualified medical personnel, if necessary.

The gateway device is preferably a mobile gateway device, and optionally and more preferably comprises a cellular telephone and/or other wireless communication device. The gateway device communicates on one hand with the sensing device, preferably a wrist-mounted device, optionally through a wire connection but more preferably through a wireless communication channel, and on the other hand with a remote location through a second wireless communication channel, such as a cellular telephone network for example.

The remote data processing server receives the data, optionally through a second cellular gateway device or directly from the mobile supplier itself using any data connection or from the Internet through an ISP (Internet Service Provider). The remote server may optionally serve a manually operated station at a call center, where the operator can view charts and histograms of up to date and historical medical measurements in order to assess the health of the subject more accurately. The station operator may optionally request the presence of medical personnel at the subject's location, if necessary.

More preferably, the data processing server automatically processes and analyzes medical information, as previously described. The server may then optionally and more preferably inform the user of the physical health status on a periodic basis and/or may optionally alert the operator, medical personnel or the subject in case of a significant deviation in one or more values of such information from a preset threshold.

Examples of medical information which may be extracted from the measured physiological parameter or parameters include, but are not limited to: heart rate; heart rate variability; breathing rate; arrhythmia of the heart (if any), as well as the general rhythm and functioning of the heart; blood pressure; presence of abnormal body movements such as convulsions for example; body position; general body movements; body temperature; presence and level of sweat; and oxygen pressure in the blood.

The system of the present invention monitors at least periodically, but more preferably continuously, or alternatively by manual activation by the user, one or more physiological parameters of the user, representing one or more physiological functions. Continuous monitoring would more easily enable the sensing device to capture more medical information about the user.

Should one or more physiological parameters be determined to be above a predefined threshold, which may represent for example such medical information as excessive heart rate, severe irregularity of heart rate, excessive body temperature or very high or low blood pressure, the remote data processing server issues a warning or an alarm relayed to the user and/or the operator and/or medical personnel. In one embodiment of the present invention where the remote data processing server is located within the gateway, the warning is issued by the gateway.

The warning and or alarm may optionally include one or more recommendations for treatments or other assistance to be provided to the user for improving the current health status of the user. Such recommendations may optionally and more preferably be displayed on an optional display of mobile gateway and/or on an optional display of sensing device. For example, if the mobile gateway includes a cellular telephone, then the display of the cellular telephone may optionally be used to display such a recommendation. Alternatively, speech or warning audio signals may optionally be delivered to the user.

According to a preferred embodiment of the present invention, the subject is preferably identified with unique identifier of the subject, such as an ID number for example. The use of a cellular telephone as a mobile gateway supports an additional, optional and preferred method for unambiguous subject authentication according to the present invention, by more preferably linking the identification of the subject (through the subject identifier) to at least one of the cellular telephone number, its SIM card number, cellular telephone serial number and IP address. The use of a cellular telephone as a mobile gateway further enables service to be provided to a plurality of subjects being members of the same group, for example a family, or a sporting team by one mobile gate.

Optionally and more preferably, the present invention also features an alarm signal for being transmitted through the mobile gateway device to the remote server in order to indicate an emergency or otherwise dangerous situation for the user. The alarm signal may optionally be triggered and transmitted according to a manual activation by the user, such as pressing an “alarm button”, which can be located on the wrist mounted sensor device or on the mobile gateway for example. Upon receipt of the manually activated alarm signal, the remote data processing server would preferably initiate immediately a call to a human operator at the call center or qualified medical personnel at the same or close to the subject location.

It should be noted that although the following discussion centers on cellular telephone networks and related wireless networks, in fact any type of communication network or combination thereof may optionally be used. For example, satellite networks are also considered to be within the scope of the present invention as an example of a type of wireless network.

Methods of establishing communication between a sensing device and a communication device by means of a gateway are also disclosed in U.S. patent application Ser. No. 10/006,357, filed on Dec. 10, 2001, having at least one inventor in common and being owned in common, which is hereby incorporated by reference as if fully set forth herein. The application discloses wrist wearable sensor capable of communicating with a gateway device preferably through a wireless communication channel. The gateway itself is in communication with a remote data processing server via the public telephone network (PSTN), the remote server carries its output processed medical data to a remote operator inhabited call center. The gateway is stationary and resides in close proximity to the subject (such as in the same room or apartment, for example, and/or otherwise in relatively close physical proximity). It allows more freedom and mobility to the subject, although it is limited by a relatively small distance between the subject and gateway. The application does not teach or suggest a gateway that supports the subject's mobility and freedom. Therefore, the present invention has a number of advantages over this previous application.

The preferred embodiment of the present invention for use with cellular communication has a number of advantages. Cellular public networks are readily available and provide a convenient and affordable method of communication. Certain standards and methods of cellular communication, such as GSM for example, support use of a single phone on an international basis. Cellular telephones have a strong CPU and a display that may be used for processing the results of measurement and display of messages addressed to the subject/owner.

The method of the present invention could also be described as a process for being performed by a data processor, and as such could optionally be implemented as software, hardware or firmware, or a combination thereof. For the present invention, a software application could be written in substantially any suitable programming language, which could easily be selected by one of ordinary skill in the art. The programming language chosen should be compatible with the computational device (computer hardware and operating system) according to which the software application is executed. Examples of suitable programming languages include, but are not limited to, Visual Basic, Visual C, standard C, C++ and Java.

### BRIEF DESCRIPTION OF THE DRAWINGS

The invention is herein described, by way of example only, with reference to the accompanying drawings, wherein:

FIG. 1 is a schematic block diagram of an exemplary embodiment of a system according to the present invention;

FIG. 2 shows a schematic block diagram of an exemplary embodiment of a sensing device according to the present invention;

FIG. 3 shows a schematic block diagram of an exemplary embodiment of a mobile gateway according to the present invention;

FIG. 4 shows a schematic block diagram of an exemplary embodiment of a receiving gateway and remote data processing server according to the present invention;

FIG. 5 is a flow chart describing an exemplary method of system initialization according to the present invention;

FIG. 6 is a flow chart describing an exemplary method of system operation in “Supervise” operation mode according to the present invention; and

FIG. 7 is a flow chart describing an exemplary method of system operation in “Alarm” operation mode according to the present invention.

### DESCRIPTION OF THE PREFERRED EMBODIMENTS

The present invention is of a system for measuring at least one physiological parameter of the user, analyzing the parameter measured and providing feedback to the user, if required. Analysis of at least one physiological parameter of the user may optionally and preferably be performed by a remotely located data processing server after transferring the physiological measured data through a gateway device.

The present invention enables such a measurement to preferably be used to directly generate medical information about the user, optionally performed by the sensing device and alternatively by the remote server. Such information may then optionally be sent via data channels to a contact center where a trained human operator may optionally examine the medical information, for example as displayed on a computer monitor.

The human operator, who may be a medically trained individual, would then preferably be able to assess the user's current medical condition from the received information. For example, such a human operator optionally and more preferably receives information from the remote data processing server. Preferably, the human operator would have access to a medical history of the user, which could optionally be stored in the database of the remote data processing server for example.

The gateway device is preferably a mobile gateway device, which communicates with the preferred but exemplary wrist-mounted device of the present invention through a wireless communication channel.

It should be noted that although the sensing device is described as being preferably wrist-mounted as a non-limiting example, in fact the sensing device may optionally (additionally or alternatively) be mounted, worn, carried or otherwise in contact with substantially any part of the body or in close proximity to the body. As another non-limiting example, the sensing device may optionally be in the form of a necklace for being worn at the neck of the subject.

The mobile gateway device is preferably an add on to a regular cellular telephone together with the cellular telephone itself or a regular cellular telephone with standard wireless access (such as Bluetooth, IEEE 802.11 protocol or IRDA), both communicates on one hand with the wrist-mounted sensing device through a wireless communication channel and on the other hand using the cellular telephone to wirelessly communicate on a separate channel with a cellular communication base station.

Examples of medical information which may optionally be extracted from the measured physiological parameter or parameters include, but are not limited to: heart rate; heart rate variability; breathing rate; arrhythmia of the heart (if any), as well as the general rhythm and functioning of the heart; blood pressure; presence of abnormal body movements such as convulsions for example; body position; general body movements; body temperature; presence and level of sweat; oxygen pressure in the blood.

The sensing device may be wrist-mounted or attached to any other part of the subject to be monitored where it ensures reliable measurement and/or monitoring of at least one physiological parameter, yet which does not inconvenience the subject. The sensing device more preferably has processing capabilities. The sensing device optionally and more preferably initiates automatic alarm signal transmission to the remote server using the gateway, if necessary, after processing the data. Optionally, the alarm signal may be given to the user and/or family members or other caregiver and/or a medical personnel, additionally or alternatively, for example by sending an SMS message to their cellular telephone or by a voice message.

According to an exemplary embodiment of the present invention, the wrist-mounted sensing device preferably features one or more sensors attached to adhesive pads, a wristband or other fastening article. The sensor(s) are preferably connected to a controlling/processing unit. With regard to data analysis, the processing unit may optionally be part of sensing device or optionally may be part of the mobile gateway or optionally may be part of the remote server. However, there is preferably a controlling unit at the sensing device for controlling the operation of the sensing device. Optionally, the sensing device may feature only an analog sensor and a transmitter without a controlling unit.

If present at the sensing device, as preferred, the processing unit more preferably supports manual and/or automatic collection of the measurement of the at least one physiological parameter; also more preferably, the processing unit is able to execute one or more instructions for extracting medical information about the user from such measurement(s).

The processing unit of the wrist-mounted sensing device may optionally control a communication unit. The communication unit of wrist-mounted sensing device preferably supports bi-directional wireless communication with the mobile gateway for transmitting the extracted information. Such communication may optionally include the raw data and any received commands from the remote data processing server. Optionally and alternatively, such communication may be unidirectional from the sensor device to the mobile gateway, such that the device cannot receive any commands from the remote server.

The mobile gateway device then preferably relays such information to a remote data processing server, which more preferably is able to automatically analyze such information and optionally provide it to a call center where the data processing server is preferably located. At such a call center, a human operator who may be medical personnel can see this information on a computer monitor and talk to the patient using the cellular (mobile) gateway as a regular telephone. The operator alternatively or additionally may receive the information at any other location with LAN connection or Internet connection to the remote server using a computer with such a communication capabilities, or alternatively using methods such as sending a message according to at least one of a SMS (Short Message Service) or MMS (Multi Media Service), or electronic mail such as an e-mail message or facsimile, or Internet based messaging (such as in cellular telephones operating in GPRS networks), to the cellular telephone of medical personnel. Therefore, continuous monitoring of the medical information and/or physiological parameters of the user may optionally and more preferably be made, enabling better medical care for the user.

If the mobile gateway optionally and more preferably features a built in or add-on videocamera (such in the case of 3G (third generation) cellular telephones), a visual and audio conversation could be made between the call center operator and the patient (user).

According to one embodiment of the present invention, the mobile gateway preferably comprises a wireless communication transceiver, a controlling, a processing unit for processing the received data and another communication unit for communication with the cellular telephone, in which the mobile gateway preferably resides and/or to which the mobile gateway is preferably attached.

According to another embodiment of the present invention, the mobile gateway comprises a wireless communication transceiver, another communication unit for communication with the cellular telephone and a cellular telephone. The wireless communication transceiver may communicate with the sensor device. In addition, processing and controlling units of the cellular telephone may optionally be used for processing the sensing device outputs and for relaying the processed data to the remote server.

According to still another embodiment of the present invention, the mobile gateway may optionally be implemented as a cellular telephone for performing the communication and processing functions of the mobile gateway according to the present invention, such that these functions (and the components required to support these functions) are preferably incorporated entirely within the cellular telephone itself.

According to yet another embodiment of the present invention, the mobile gateway may optionally be implemented as part of the sensing device for performing the communication and processing functions of the mobile gateway according to the present invention, such that these functions (and the components required to support these functions) are preferably incorporated entirely within the sensing device itself. Optionally, this implementation may include SMS messaging and the ability to establish a voice conversation between the patient and the call center.

According to one embodiment of the present invention, the communication link between the mobile gateway and the remote data processing server optionally and more preferably comprise one or more of the following methods, although the communication is not limited to these methods: Short Message Service (SMS) messages, MMS, Internet messaging, and Java applets. Such communication is bi-directional, for example in order to allow the remote server to send one or more reminders or other messages to be displayed on the cellular telephone display about various medical matters, such as for assisting patient compliance with drug dosing regimes, reminding the subject to take medication, visits to medical personnel and so forth. Optionally, such communication can be performed unidirectionally from the device to the remote server.

Additionally, the communication link between the sensing device and the mobile gateway may use Bluetooth protocol, IEEE 802.11, Home RF protocol or alternatively any other proprietary protocol using a RF link, any type of wireless infrared, contact less technology or any other radio frequency protocol communication link.

According to a further embodiment of the present invention, there is provided a call center with wired or wireless data link connection to the remote data processing server. The call center human operator may optionally and more preferably receive an automatically generated diagnosis from such a server. The operator then preferably determines one or more actions to be performed according to the diagnosis.

According to yet another embodiment of the present invention, the remote server is located inside the mobile gateway, such that the diagnostic and/or other analysis procedures (for obtaining diagnostic and/or other types of information) are performed more preferably only for users who own that mobile gateway. The gateway optionally communicates with the call center in case of medical emergency. The mobile gateway optionally may use the cellular telephone's display to present messages to the user. This can be made in GSM telephones by using its SIM (Subscriber Identity Module) with methods such as the SIM tool kit, which enables the SIM to run small software programs.

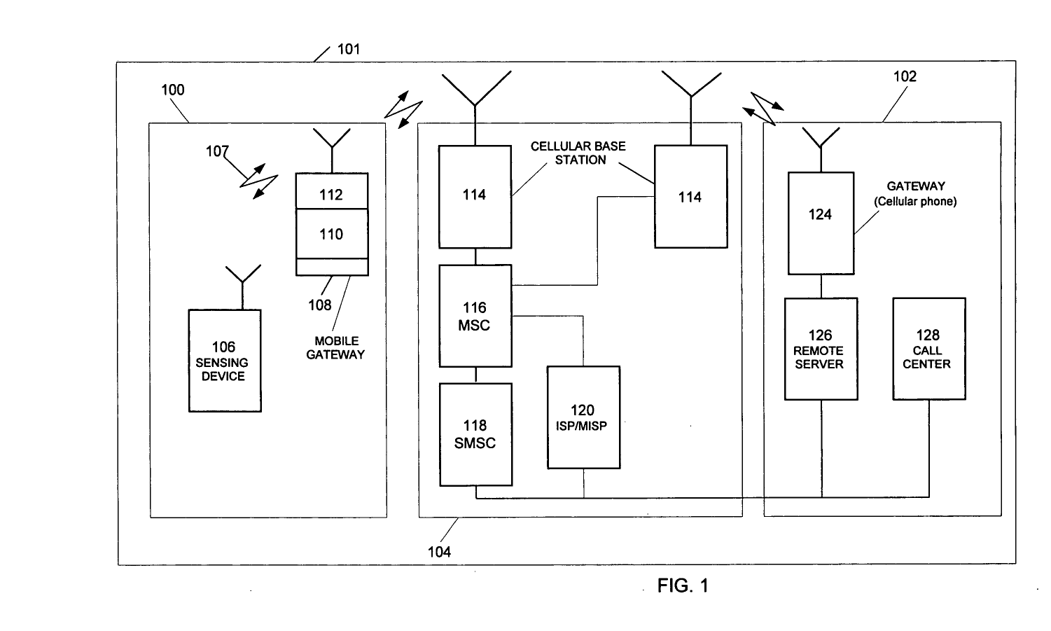

Referring now to the drawings, FIG. 1 is a schematic block diagram of an exemplary system according to the present invention. As shown, a system 101 features a sensing and transmitting sub-system 100, a receiving and processing sub-system 102 and existing communication infrastructure 104, such as cellular or land telephone lines, or any other type of wireless and/or wired infrastructure.

Sensing and transmitting sub-system 100 features a sensing device 106, for measuring at least one physiological parameter. Sensing device 106 may optionally be a wearable device to be worn by a user, optionally and more preferably as a wrist-mounted device, for example by being attached with adhesive pads, a wristband or other fastening article to the wrist of the user. Alternatively, sensing device 106 may optionally be attached to any other part of the subject to be monitored, more preferably for being able to reliably measure one or more physiological parameters, while also more preferably not inconveniencing the subject.

Sensing device 106 communicates preferably through a bi-directional, wireless communication link 107 with at least mobile gateway device 108. As previously described, communication is optionally and alternatively unidirectional. Mobile gateway device 108 may optionally comprise a cellular telephone 112 and/or an add-on to cellular telephone device 110. Cellular telephone 112 may be such as Nokia 6300 series commercially available from Nokia, Finland or such as SGH R210 commercially available from Samsung, South Korea or any other suitable cellular telephone. The operation of an add-on to cellular telephone device 110 is described below.

Optionally, system 101 may comprise multiple sensing devices 106 and multiple mobile gateway devices 108.

Mobile gateway device 108 preferably communicates through existing cellular infrastructure, which may optionally be a network of cellular telephone base stations 114 for example, of which two are shown for descriptive purposes only and without any intention of being limiting. Mobile gateway device 108 more preferably also communicates with a fixed gateway device 124 using SMS messages. Fixed gateway device 124 is also preferably located at receiving and processing sub-system 102. Fixed gateway device 124 is preferably, but not necessarily, a cellular telephone similar to cellular telephone 112 of mobile gateway device 108. Fixed gateway device 124 optionally and more preferably communicates for example with the help of a hard wired F/M—bus and/or any other suitable hardware bus, preferably with remote data processing server 126.

Such communication is preferably performed directly with a data processing server 126. Both fixed gateway device 124 and remote data processing server 126 are preferably remotely located with respect to sensing device 106 and mobile gateway device 108.

Cellular base stations 114 are optionally and more preferably implemented as conventional cellular base stations operating in accordance with well known cellular communication standards such as CDMA, GSM, TDMA, GPRS, 3G (Third Generation) systems and so forth. Cellular infrastructure may preferably include a Mobile Services Switching Center (MSC) 116 and Short Message Service Center (SMSC) 118. Only two cellular base stations 114 are shown for the purposes of illustration only and without any intention of being limiting. These two cellular base stations 114 govern the communication process between mobile gateway device 108 and fixed gateway device 124 for at least a certain period of time. Cellular base station 114 preferably communicates with fixed gateway device 124 and/or remote data processing server 126 through a SMS messages based data link.

Transmitted data may optionally and more preferably be communicated by using a Short Message Service (SMS). For this type of communication, transmitted data would preferably be passed from Mobile Message Switching Services Center (MSC) 116 to a Short Message Service center (SMSC) 118. SMSC 118 would then preferably transmit data in the form of an SMS message to local cellular base station 114, which is preferably nearby remote data processing server 126. The SMS is then received and transferred to remote data processing server 126. Of course, such communication is also optionally and more preferably bidirectional. Alternately, SMSC 118 can send the text of the SMS message in an Internet format (such as TCP/IP protocol) to the remote processing server using the Internet.

Additionally or alternatively, mobile gateway can establish a dial-up connection with an ISP (Internet Service Provider) 120 in order to communicate with the remote server using an Internet format. Data may be optionally transmitted from mobile gateway device 108 to local cellular base station 114 and then to MSC 116, then through a suitable communication infrastructure, such as a direct dial up line or LAN to ISP (Internet service provider) 120, for example. ISP 120 may optionally be implemented as a MISP (mobile Internet service provider) for example. From ISP 120, an Internet connection would be established with remote data processing server 126 using standard TCP/IP protocol or any other suitable protocol, for delivering any data to remote data processing server 126.

Receiving and processing sub-system 102 may optionally and more preferably include a manually operated call center 128. Call center 128 may optionally and preferably perform such functions as analyzing data, providing a telephone communication and/or video conferencing channel between the operator and the patient and also for optionally enabling medical personnel to more easily view and access the medical information.

According to one of the exemplary embodiments of the present invention, mobile gateway device 108 may be a wireless communication transceiver, such as a cellular telephone 112 or an add-on to cellular telephone device 110. The use of a cellular telephone as a gateway device has a number of advantages. A subject who owns a cellular telephone may be expected to carry it on at least a regular basis. Hence, use of a cellular telephone as a communication gateway does not limit subject movement to a certain radius as in case of a static communication gateway.

Also, the use of a cellular telephone as a communication gateway does not force the subject to handle the cellular telephone in order for the device to be operative as a communication gateway, as mere proximity to the cellular telephone may be sufficient. This proximity may be derived from regular use of cellular telephone 112 as for any cellular telephone. Sensing device 106 is preferably implemented such that this proximity is sufficient for enabling communication with cellular telephone 112 (a distance of up to about 50 meters is preferable, although a particular distance is not a requirement for the operation of the present invention).

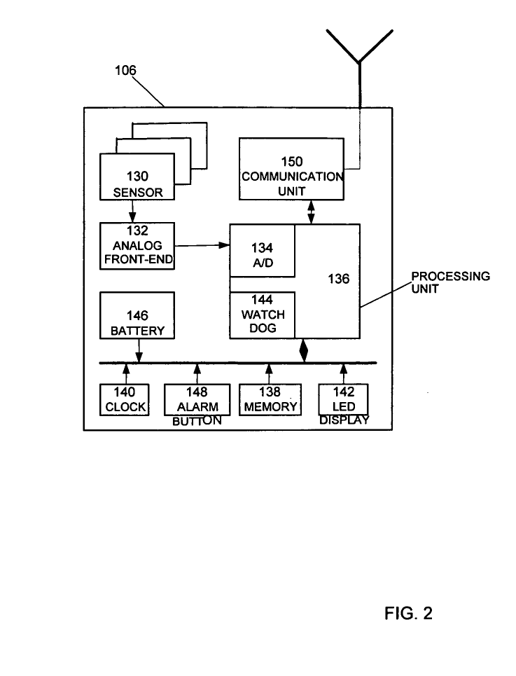

The function and structure of an exemplary sensing device 106 is described in greater detail below. FIG. 2 shows a schematic block diagram of an exemplary embodiment of a sensing device 106, which is a part of the present invention. Device 106 preferably features at least one physiological parameter sensor 130 for measuring at least one physiological parameter of the user, of which a plurality are shown for the purposes of discussion only and without any intention of being limiting. The physiological parameters of the subject may be blood pressure pulses (heart rhythms), body movements, temperature, blood pressure, and others. Sensors 130 may be part of sensing device 106 and may be an attachment to sensing device 106.

An exemplary physiological sensor 130 may be a piezoceramic sensor, which is in direct contact with the skin of the user. For this non-limiting example, sensor 130 preferably generates an electrical signal, having amplitude corresponding to the magnitude of applied pressure. Therefore, if at least a portion of the transducer is located adjacent to, and in physical contact with, an area of the wrist where blood pressure pulses may be detected. Alternately, the pressure may be delivered by the fastening article from the artery to the transducer. Sensor 130 generates electrical pressure pulses corresponding to the detected blood pressure pulses. Each of the electrical pressure pulses preferably defines a maximum voltage over a systolic interval and a minimum voltage over a diastolic interval.

The output of sensor 130 is preferably an analog signal. Analog front end 132 preferably receives and amplifies and filters the signal to a level supporting reliable analog to digital conversion within the desirable frequency band pass. Analog to Digital converter 134 then converts this signal into digital form. Processing unit 136 optionally and more preferably receives and processes the digital signal containing measured physiological data. Processing unit 136 preferably executes at least one instruction for processing the data obtained by sensor 130; alternatively, processing unit 136 only delivers the received raw data to mobile gateway device 108.

Sensing device 106 optionally and preferably features an internal communication unit 150, for preferably performing bidirectional communication with mobile gateway device 108. Optionally and alternatively, internal communication unit 150 provides unidirectional communication for transmission of data only from sensing device 106 to mobile gateway device 108. Processing unit 136 preferably feeds a communication unit 150 with processed data or raw data obtained from sensor or sensors 130.

According to the present invention, the bidirectional communication with communication unit 150 of sensing device 102 may optionally use any suitable RF (Radio Frequency) frequency, such as 433.92 MHz radio frequency for example, Bluetooth protocol or any other approved wireless communication standard.

According to preferred embodiments of the present invention, sensor device 106 also optionally and more preferably comprises at least one or more of a memory 138, a real time clock 140, and an indicator display 142, for example in order to indicate an alert activation or a low battery level.

A battery 146 optionally and more preferably provides power supply to sensing device 106. Sensing device 106 may also optionally and preferably feature a manually operated alarm button 148 to be manually activated by the user, for example if the user is in distress.

A “watch dog” type arrangement 144 may optionally and preferably be used to watch for any software and/or hardware malfunction and to reset the device in such a case. An example for the operation of “watch dog” type arrangement 144 is as follows. First, “watch dog” type arrangement 144 preferably triggers a watchdog function shown as a “Watchdog” process.

If the end of a watchdog time period is reached, sensor device 106 is assumed to have a fault in its operation, and a master reset is preferably initiated automatically. The end of such a time period is reached if sensor device 106 does not triggered automatically or manually from sleep mode. Examples of such triggers include but are not limited to, pressing a panic button manually by the user; and signaling by real time clock 140 which indicates the need for sensor device 106 to execute an automatic check.

Real time clock 140 more preferably provides an accurate time and date for each measurement, as sensing device 106 can optionally store a few measurements before transmitting such data and/or information to mobile gateway device 108. Real time clock 140 may also optionally be used for such applications as reminding the subject to take medication, perform a medical diagnostic measurement, and so forth. A/D converter 134 may support multiple inputs in order to convert the analog signals collected by different sensors 130 into a digital signal.

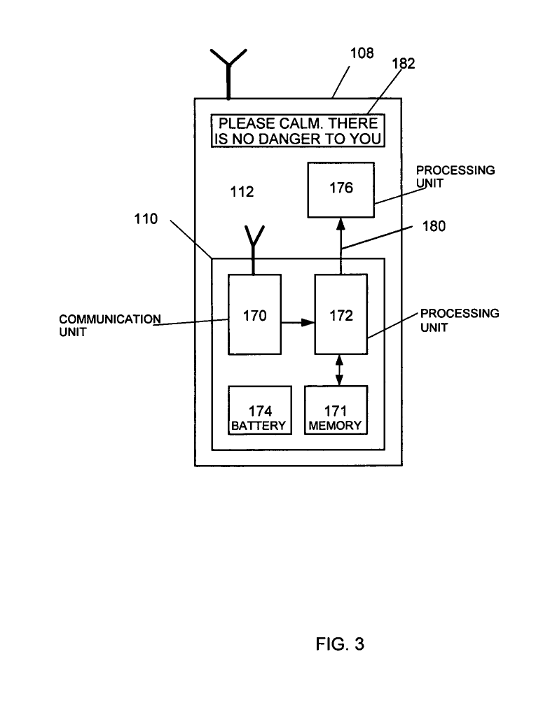

According to the present invention, the optional implementation of communication gateway as an add-on to cellular telephone device and communicating with cellular telephone has most of the cellular telephone advantages, but does not need to be packaged within a particular space within a telephone. FIG. 3 shows a schematic block diagram of an exemplary embodiment of a mobile gateway according to the present invention. The particular embodiment of mobile communication gateway 108 includes a cellular telephone 112 and add-on device 110. Add-on device 110 may optionally be an accessory which is optionally and preferably attached and/or connected to the cellular telephone package, or may optionally be carried separately from cellular telephone 112. One example of an implementation for such an accessory optionally and preferably includes at least an electrical connection to cellular telephone 112, for example through an external data connector, but may also optionally, additionally or alternatively, include a direct wired connection to the communication port of processing unit 176 of cellular telephone 112.

Alternately, the communication can be established using an infrared data link (communication according to infrared signals), using an Infrared port of cellular telephone 112, if present. Such a port may optionally be located within cellular telephone 112, as a hardware component thereof.

Add-on device 110 optionally and preferably includes a communication unit 170, and a processing unit 172. Add-on device 110 may use the cellular telephone battery (not shown) as a source of power but preferably and for safety reasons, add-on device 110 may optionally include a dedicated battery 174 and/or other power source (not shown). Battery 174 is preferably operative all of the time, or alternatively becomes operative when the cellular telephone battery is exhausted. The provision of an additional power sources provides for maintenance of communications with sensing device (not shown), storing the received data into a local memory 171 until cellular telephone 112 regains power, then add-on device 110 preferably sends the accumulated data to the remote server (not shown, see FIG. 1).

Communication unit 170 of add-on device 110 preferably communicates with communication unit 150 of sensing device 106 using some type of RF (radio frequency) signal, such as 433 MHz RF for example, Bluetooth protocol or any other approved wireless communication standard. Communication unit 170 feeds the received data into processing unit 172.

Processing unit 172 communicates further with processing unit 176 of cellular telephone 112. Communication between processing unit 172 and processing unit 176 may optionally be enabled by wireless means or by a connection of bus cable (such as F/M bus of NOKIA phones) or any other modem type connection 180 or UART (universal asynchronous receive/transmit) protocol enabled direct-wired connection. UART enabled connections are intended for serial communication between processing units 172,176. Infrared and/or other types of wireless connections are also optionally possible.

Cellular telephone display 182 may optionally display, for the convenience of the user, a copy of messages transmitted by cellular telephone 112 to remote data processing server 126 and/or a message from remote data processing server 126 to the user.

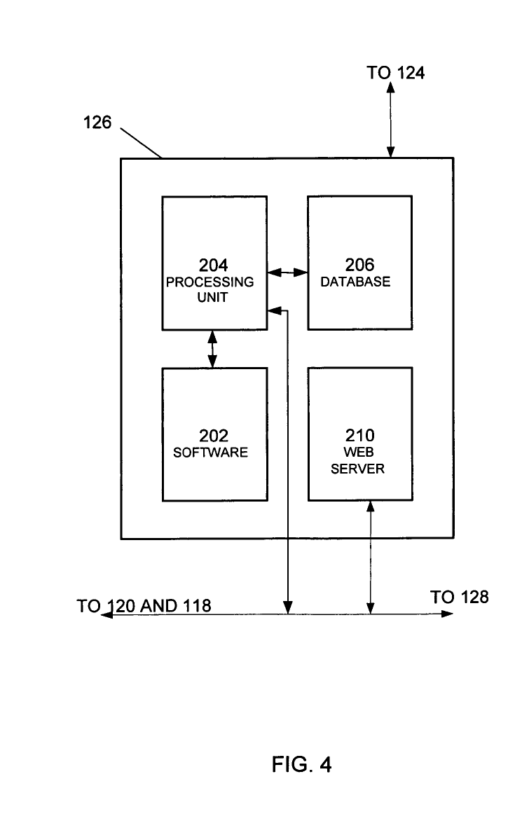

Attention is drawn now to receiving gateway 124 and remote data processing server 126. FIG. 4 shows a schematic block diagram of an exemplary embodiment of a receiving gateway and remote data processing server, according to the present invention. In the particular exemplary embodiment, receiving gateway 124 is a cellular telephone similar to one used in mobile gateway device 108.

Remote data processing server 126 preferably features a software program 202 for automatically performed (non-manual) diagnostic algorithms. Remote data processing server 126 also preferably features a processing unit 204 for processing data received from gateway device 124, and a database 206 for storing raw and or processed data and the user's health history. A Web server 210 for enabling provision of data and/or other types of user interactions through Web pages (according to the HTTP (hyper-text transfer protocol) protocol) may also optionally be connected to ISP 120. In any case, access to data in database 206 is preferably secured, for example through a firewall which limits such access to authorized users.

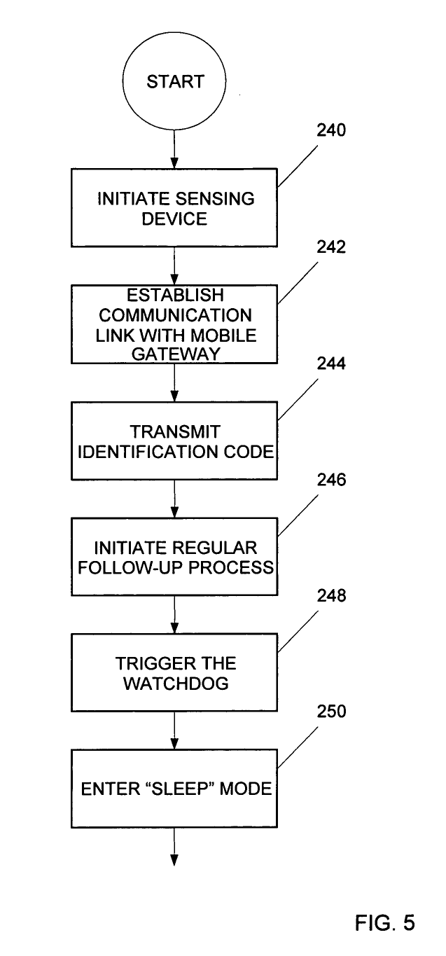

The method of operation of monitoring system for assessing physiological parameters of a subject is described with regard to FIG. 5, which is a flow chart of the first stages of system initialization. As sensing device 106 (not shown) begins operation for the first time, the software of device 106 at step 240 preferably makes some initializations using default values. Communication link with mobile gateway 114 (not shown) is preferably established at step 242. An identification code is optionally transmitted to remote data processing server 126 (not shown) at step 244 via mobile gateway device 108 (not shown). Remote data processing server 126 more preferably identifies the new subscriber, creates a respective database entry and initiates the regular follow-up process at step 246. The process of identification optionally and more preferably includes the provision of a subscriber identifier, and most preferably includes information related to mobile gateway 114 (not shown) as previously described.

Once sensing device 106 has been initialized and the subscriber is identified, more preferably also including sending an acknowledgement message by remote data processing server 126, the software at step 248 preferably triggers a watchdog function, and then at step 250 enters a “sleeping” mode for saving battery life. If the end of a watchdog time period is reached, the device is assumed to have a fault in its operation and a master reset is preferably initiated automatically.

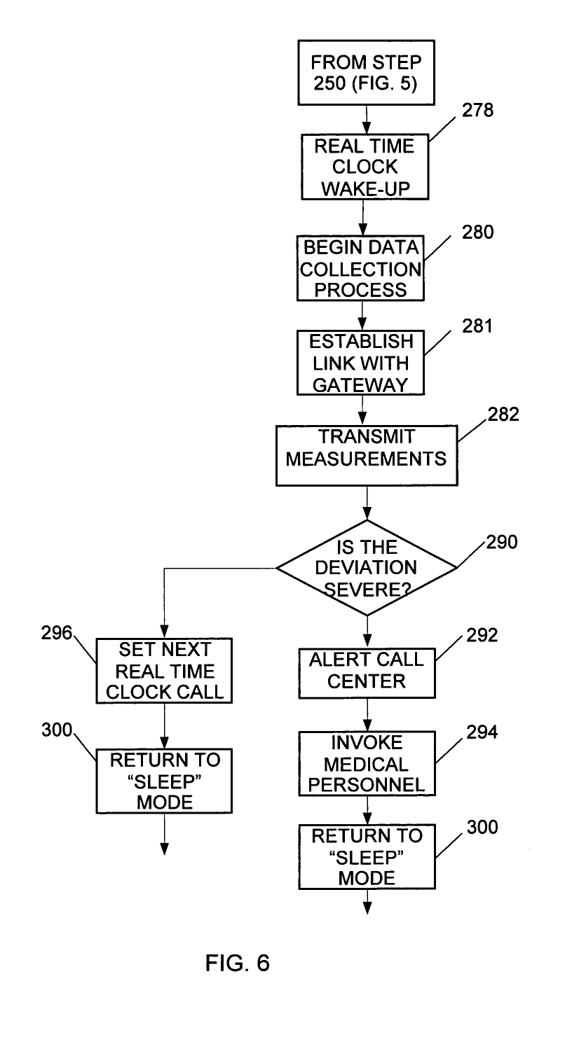

There may optionally be a number of operation modes or “wake-up” triggers from the “sleeping” mode. FIG. 6 is a flow chart describing a method of system operation in “measurement” mode. In “measurement” operation mode, real time clock 140 (not shown) wakes up sensing device 106 (not shown) at step 278. A data collection process is initiated at step 280, by processing unit 136 (not shown). Sensing device 106 (not shown) then initiates communication with the mobile gateway in step 281, and transmits measurements in step 282.

Remote data processing server 126 can initiate an “automatic alarm” mode, if following the analysis or the information at step 290, remote data processing server 126 discovers that at least one measured parameter exceeds a predefined threshold at least once, remote data processing server 126 preferably issues a command to the sensing device 106. Remote data processing server 126 preferably defines the severity of the parameter deviation. If the deviation of the parameter is severe (i.e. statistically and/or medically significant), remote data processing server 126 optionally and more preferably contacts call center 128. Call center 128 may optionally initiate a voice communication with the subject at step 292, and/or may optionally directly contact medical personnel at step 294.

If the deviation in the measured parameter is not severe, at step 296, remote data processing server 126 initiates a prioritized subject follow-up process. The prioritized follow-up process may contain such commands as an increased frequency of measurement of the parameters and/or communication of these measurements. If the deviation of the parameter measurement does not persist, and more preferably if no other parameters exceed their thresholds, at step 300 remote data processing server 126 preferably switches/returns measuring device to normal “Supervise” timing mode.

Sensing unit 106 performs the command and sends an ACK (acknowledgement message) remote data processing server 126 through the mobile gateway device 108. Remote data processing server 126 returns an ACK with another command to continue or without a command to terminate this process. After performing the last command, sensing device 106 returns to the “Sleep” state.

Remote data processing server may send the above commands to sensing device 106 using wireless gateway 108. Fixed gateway 124 relays this data/command message to mobile gateway device 108, optionally and preferably by using methods such as a SMS or similar methods. Mobile gateway device 108 preferably sends these data and/or commands to sensing device 106, preferably through a wireless link established between them. Remote data processing server 126, following signal analysis at step 290, preferably decides on the content and type of the message sent to the subject.

There may be cases where the acknowledge message either from mobile gateway device 108 or remote data processing station 126 is not received within a set time period. After the expiration of this time period, sensing device 106 optionally and preferably initiates a repeated message transmission. Additional transmissions are initiated, if necessary. However, if after a predefined number of repeated times an acknowledge message is not received, an error message is stored within a log and no more tries are made. More preferably, an indicator LED 142 starts blinking for a few seconds, optionally with an audible alarm. Then, the process returns to the “Sleep” state.

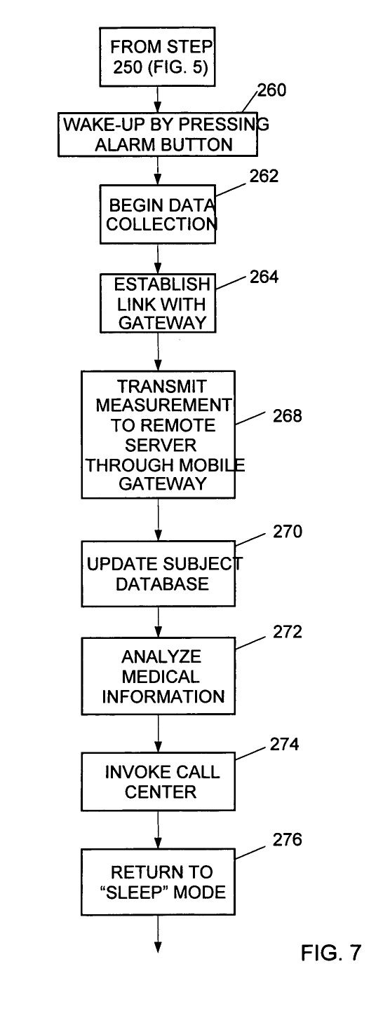

FIG. 7 is a flow chart describing a method of system operation in “Alarm” operation mode. In “Alarm” operation mode, sensing device 106 (not shown) typically wakes-up at step 260 on pressing an alarm button. Following this “wake-up” trigger, the process preferably moves to the “Supervise” state, where sensing device 106 at step 262 collects data from sensors 130 (not shown; see FIG. 2). At step 264, sensing device 106 (not shown) then initiates communication with the mobile gateway (mobile gateway device 108, not shown).

At step 268, communication unit 150 preferably enters the “TX/RX” (Transmit/Receive) state, where communication unit 150 transmits a distress message containing the identifier, and the collected data and/or processed medical parameters to mobile gateway device 108. Then sensing device 106 preferably enters a receiving mode for a few seconds, waiting for acknowledge (ACK) from remote data processing server 126 through mobile gateway device 108.

Mobile gateway device 108, at step 268, preferably relays the distress message containing the identifier and the collected data and/or processed medical parameters to remote data processing server 126, for example through cellular base stations 114 by using fixed gateway device 124, and/or through a dial up (telephonic) connection to ISP/MISP, and/or SMS message transmission from the SMSC as previously described. Optionally and additionally, if communication unit 150 is a cellular telephone, the message may contain information on any one or more of the cellular telephone number of the user, the cellular telephone SIM card number and the current location (for example, according to the cellular base station to which mobile gateway device 108 made a call). Such information can optionally be obtained by acquiring the cellular telephone of mobile gateway device 108 with appropriate AT command.

Remote data processing server 126 at step 270 processes the message and stores it on a database 206 (not shown; see FIG. 4). Then, remote data processing server 126 returns an ACK to sensing device 106 with or without a command. In case of a return command, sensing device 106 preferably sends back an acknowledgement message. Remote data processing server 126 returns to sensing device 106 more preferably only if remote data processing server 126 is to send another command, and so on. After performing the last command, sensing device 106 preferably returns to the “Sleep” state.

In step 272, remote data processing server 126 preferably analyzes the medical information, for example in order to automatically determine a diagnosis.

After receiving an “Alarm” type message, remote data processing server 126 more preferably allocates a higher priority to that subject, and a human operator at call center is preferably alerted. A pop up message preferably appears on the operator's monitor, more preferably information related to the past medical history and/or current information about the user. Examples of such information include but are not limited to, the user's name, address and the current measurements as received by the message, the user's medical history such as any known diseases and/or other medical conditions, any medication being taken, and graphs or other displays of received measurements acquired from the database which may show whether the condition of the user has become degraded from previous measurements. The human operator then preferably contacts the user, for example more preferably by using the cellular telephone or fixed line telephone of the user, in order to be able to assess the situation properly.

In the third optional mode of the trigger, sensing device 106 receives a command to enter “Maintenance” using for example a dedicated switch or automatic battery low indicator. The “Maintenance” mode may optionally be initiated for technical reasons such as system maintenance, diagnostics or loading of new software (for example with the release of a new software version). Such new software may optionally be provided through a centralized mechanism to each subscriber of the services.

Some other operation processes that may optionally be treated as maintenance processes may occur in the course of regular system operation. For example each time sensing device 106 sends a message to mobile gateway device 108, a special counter preferably counts the number of transmissions made from the time of last battery replacement. Then, after a preset number of times, a low battery indicator is more preferably set. The message may optionally contain a Battery OK/Battery Low indication for the battery situation. This signal preferably appears three months (or another predefined time period) before the battery finishes, which provides sufficient time for the user to be able to replace the battery.

It will be appreciated that the above descriptions are intended only to serve as examples, and that many other embodiments are possible within the spirit and the scope of the present invention.

1. A system for obtaining information about at least one physiological function of a subject, comprising:
(a) a sensing device for measuring and transmitting the at least one physiological parameter to form a transmitted measurement, wherein at least a portion of said sensing device is in direct physical contact with at least a portion of the subject;
(b) a mobile gateway for receiving said transmitted measurement; and
(c) a remote data processing server for receiving said transmitted measurement from said mobile gateway and for at least assisting in an analysis process for analyzing said transmitted measurement, wherein said analysis process provides the information about the at least one physiological function of the subject.

2. The system of claim 1, wherein said remote data processing server further comprises a processing unit for automatically performing said analysis process.

3. The system of claim 2, wherein said analysis process includes a diagnostic process, such that said analysis process includes automatically producing a diagnosis.

4. The system of claim 1, wherein said mobile gateway is at least partially in wireless communication with remote data processing server.

5. The system of claim 4, wherein said mobile gateway further comprises a wireless communication device, wherein components of said mobile gateway, apart from said wireless communication device, are connected to said wireless communication device as add-on components.

6. The system of claim 5, wherein said wireless communication device comprises a cellular telephone.

7. The system of claim 4, wherein said mobile gateway further comprises a cellular telephone for said wireless communication.

8. The system of claim 5, further comprising:
(d) a fixed gateway for being in wireless communication with said cellular telephone, said fixed gateway also being in communication with said remote data processing server.

9. The system of claim 8, wherein said fixed gateway is in wireless communication with said cellular telephone according to a SMS (short message system) messaging protocol.

10. The system of claim 9, further comprising:
(e) an SMSC (SMS center) for supporting said wireless communication according to said SMS messaging protocol.

11. The system of claim 1, wherein communication between said mobile gateway and said remote data processing server is bi-directional.

12. The system of claim 1, wherein said remote data processing server provides said transmitted measurement to a human operator for manually performing said analysis process.

13. The system of claim 1, wherein said remote data processing server partially performs said analysis process and wherein at least a portion of said analysis process is performed by a human operator.

14. The system of claim 1, further comprising:
(d) a fixed gateway in communication with said remote data processing server and in communication with said mobile gateway device, wherein said fixed gateway device receives said transmitted measurement from said mobile gateway device and provides said transmitted measurement to said remote data processing server, wherein said fixed gateway is connected to said remote data processing server through a fixed connection.

15. The system of claim 1, wherein said sensing device further comprises a sensor for measuring at least one physiological parameter of the subject, said sensor being in contact with at least a portion of the subject, and a processor for controlling at least one function of said sensor.

16. The system of claim 15, wherein said processor of said sensing device receives a signal from said sensor and converts said at least one measurement to form medical information.

17. The system of claim 1, wherein said sensing device further comprises a fastening article for being fastened to a wrist of the subject.

18. The system of claim 17, wherein said fastening article comprises an adhesive pad for being attached to the skin of the subject.

19. The system of claim 1, wherein said sensing device further comprises a communication unit for transmitting said medical information to said mobile gateway.

20. The system of claim 19, wherein said communication unit communicates through wireless communication.

21. The system of claim 20, wherein said wireless communication is performed according to at least one of Bluetooth communication, RF (radio frequency) communication and infra-red communication.

22. The system of claim 1, wherein said mobile gateway further comprises a processing unit for performing said analysis process for determining the information about the at least one physiological function of the subject.

23. The system of claim 1, further comprising:
(d) a display for displaying the information about the at least one physiological function of the subject to an operator; and
wherein said mobile gateway further comprises a camera and a mobile telephone for performing a visual and audio conversation between said operator and the subject.

24. The system of claim 1, further comprising:
(d) a database for storing said transmitted measurement and for being connected to said remote data processing server.

25. The system of claim 24, wherein at least said transmitted measurement is retrievable from said database for at least one of analysis and display.

26. The system of claim 24, further comprising:
(e) a Web server for communicating with said remote data processing server and for serving data from said database through a Web-based function.

27. The system of claim 1, further comprising:
(d) a videocamera in communication with said cellular telephone, for supporting a videoconference with the subject.

28. A method for obtaining information about at least one physiological function of a subject, comprising:
measuring the at least one physiological parameter at the subject by a sensing device, wherein at least a portion of said sensing device is at least in close physical proximity to at least a portion of the subject;
transmitting said measurement to form a transmitted measurement;
receiving said transmitted measurement by a mobile gateway, wherein said mobile gateway is capable of further transmitting said transmitted measurement.

29. The method of claim 28, wherein said transmitted measurement is transmitted to a data processing server, the method further comprising:
receiving said transmitted measurement from said mobile gateway by said data processing server; and
at least assisting in an analysis process for analyzing said transmitted measurement, wherein said analysis process provides the information about the at least one physiological function of the subject.

30. The method of claim 29, further comprising:
identifying the subject according to a subject identifier, for securing said transmitted measurement.

31. The method of claim 30, wherein said subject identifier is at least partially determined according to at least one characteristic of said mobile gateway.

32. The method of claim 31, wherein said mobile gateway includes a cellular telephone, and wherein said at least one characteristic of said cellular telephone is selected from the group consisting of a telephone number, a SIM card number, a cellular telephone serial number and an IP (Internet Protocol) address.

33. The method of claim 28, further comprising:
storing said transmitted measurement and the information about the at least one physiological function of the subject to form a medical history of the subject.

34. The method of claim 33, wherein recently transmitted measurements are compared to said medical history to determine if at least one physiological function is deteriorating.

35. The method of claim 34, wherein if said at least one physiological function is deteriorating, at least one medically trained individual is alerted.

36. The method of claim 29, wherein said analysis process further comprises automatically performing a diagnosis of the subject.

37. The method of claim 28, wherein said analysis process further comprises displaying the information about the at least one physiological function of the subject to medical personnel.

38. The method of claim 28, wherein said portion of said sensing device is in direct physical contact with said portion of the subject.

39. A system for obtaining information about at least one physiological function of a subject, comprising:
(a) a sensing device for measuring and transmitting the at least one physiological parameter to form a transmitted measurement, wherein at least a portion of said sensing device is in direct physical contact with at least a portion of the subject;
(b) a mobile gateway for receiving said transmitted measurement from said sensing device according to wireless communication; and
(c) a remote data processing server for receiving said transmitted measurement from said mobile gateway at least partially according to wireless communication and for at least assisting in an analysis process for analyzing said transmitted measurement, wherein said analysis process provides the information about the at least one physiological function of the subject.

40. A system for obtaining information about at least one physiological function of a subject, comprising:
(a) a sensing device for measuring and transmitting the at least one physiological parameter to form a transmitted measurement, wherein said sensing device is not in direct physical contact with the subject;
(b) a mobile gateway for receiving said transmitted measurement; and
(c) a remote data processing server for receiving said transmitted measurement from said mobile gateway and for at least assisting in an analysis process for analyzing said transmitted measurement, wherein said analysis process provides the information about the at least one physiological function of the subject.

41. A system for transmitting medical information about a subject to a remote location, the medical information being obtained from at least one physiological measurement of the subject, the system comprising:
(a) a cellular telephone in physical proximity to the subject for receiving the at least one physiological measurement of the subject; and
(b) a data processing unit contained within said cellular telephone for processing the at least one physiological measurement of the subject to form the medical information, such that said cellular telephone is a local server for the medical information.

42. The system of claim 41, further comprising:
(c) a videocamera in communication with said cellular telephone, for supporting a videoconference with the subject.

43. A system for obtaining information about at least one physiological function of a subject, comprising:
(a) a sensing device for measuring and transmitting the at least one physiological parameter to form a transmitted measurement, wherein at least a portion of said sensing device is in direct physical contact with at least a portion of the subject;
(b) a mobile gateway for receiving said transmitted measurement and for performing an analysis process for determining the information about the at least one physiological function of the subject;
(c) a remote data processing server for receiving the information about the at least one physiological function of the subject; and
(d) a database in communication with said remote data processing server, wherein the information about the at least one physiological function of the subject is stored in said database by said remote data processing server.
